(19) United States
(12) Patent Application Publication
(10) Pub. No.: US 2010/0266990 A1
(43) Pub. Date: Oct. 21, 2010

(54) DENTAL ANTIMICROBIAL FORMULATIONS AND METHODS OF USING THE SAME

(76) Inventors: Marianna Cooley, Houston, TX (US); Mikki Lease Fraser, Reno, NV (US)

Correspondence Address: Nielsen IP Law LLC, 1177 West Loop South, Suite 1600, Houston, TX, 77027, US

(21) Appl. No.: 12/520,753

(22) PCT Filed: Dec. 22, 2006
(86) PCT No.: PCT/US06/62549

§ 371 Date: May 19, 2010

(51) Int. Cl. A61C 5/00; A61K 8/19; A61Q 11/00; A61K 6/00; A61K 6/02; A61C 19/06

(52) U.S. Cl. 433/228.1; 424/49; 106/35; 433/215

(57) ABSTRACT

An antimicrobial formulation for dental applications includes colloidal silver, from between about 0.01 to 2 percent by weight and colloidal copper, from between about 0.05 to about 10 percent by weight. The antimicrobial formulations are used to treat microorganisms on a tooth to prevent tooth decay by administering an effective amount of the antimicrobial formulation alone or in combination with a dental varnish, stannous fluoride, or sodium fluoride. The antimicrobial formulation may be part of a kit or used in a biocompatible time release chip.

### FIELD OF INVENTION

The present invention relates to antimicrobial formulations and methods of using the same, and more particularly, the present invention relates to such formulations incorporated in dental composite materials as well as use of such formulations in preventative treatments against dental bacterial accumulation.

### CROSS-REFERENCE TO RELATED APPLICATIONS

None.

### STATEMENT REGARDING FEDERALLY SPONSORED RESEARCH OR DEVELOPMENT

None.

### THE NAMES OF THE PARTIES TO A JOINT RESEARCH AGREEMENT

None.

### REFERENCE TO SEQUENCE LISTING

None.

### BACKGROUND OF THE INVENTION

Biofilms are diverse microbial colonies of bacteria, spirochetes, fungi, cocci, viruses, etc. that colonize and begin producing slime (mucopolysaccharides) that makes them invisible to the human immune system. Biofilms are resistant to immune cells and antibiotics. Microorganisms in a biofilm survive better and exhibit stronger resistance to the environment than do cells that are not in a biofilm. See e.g, Merritt et al., Bacterial Biofilm and Dentistry, CDA Journal, 29(5): 355-360 (2001)). Biofilms are the chief contributors of dental disease. Biofilms can form in various environments, including within the mouth and in water supply lines Id.

Tooth decay is caused by acid-producing bacteria that reside within biofilms. The acidic pH levels in the mouth affect teeth because of their high mineral content. Depending on the extent of tooth destruction, various treatments can be used to restore teeth to proper form, function, and esthetics, although there is no known method to regenerate large amounts of tooth structure. Instead, dental health organizations advocate preventative measures, such as regular oral hygiene, dietary modifications, and fluoride therapy to avoid forming dental caries.

Fluoride therapy is the delivery of fluoride to the teeth topically or systemically, which is designed to prevent tooth decay (dental caries) which results in cavities. Most commonly, fluoride is applied topically to the teeth using gels, varnishes, toothpaste/dentifrices or mouth rinse. Systemic delivery involves fluoride supplementation using tablets or drops which are swallowed. This type of delivery is rarely used where public water supplies are fluoridated.

Despite the benefits of such fluoride treatment, the commonly used fluoride salts, stannous and sodium fluoride, have short half lives when exposed to moisture. Thus, the benefits of fluoride treatment are compromised by the constant exposure to saliva in the mouth. A need therefore exists to for the development of dental formulations with lasting germicidal activity.

### BRIEF SUMMARY OF THE INVENTION

The present invention provides an antimicrobial formulation for dental applications which includes colloidal silver, from between about 0.001 to 2 percent by weight and colloidal copper, from between about 0.001 to about 10 percent by weight.

The formulations disclosed herein are used to treat microorganisms on a tooth by administering to a patient an effective amount of the antimicrobial formulation alone or in combination with a dental varnish, stannous fluoride, or sodium fluoride.

A method of preventing tooth decay includes administering to a patient an effective amount of an antimicrobial formulation alone or in combination with a dental varnish, stannous fluoride, or sodium fluoride.

The antimicrobial formulation may be part of a kit or used in a biocompatible time release chip.

### DETAILED DESCRIPTION OF THE INVENTION

The present invention provides for formulations containing low non-toxic amounts of colloidal copper and colloidal silver, that are effective in treating microorganisms found in biofilm and other environments. For dental indications, the formulations of the present invention retard biofilm formation, eliminates existing biofilm and may promote secondary dentin formation. Also, the antimicrobial formulation may be used to treat the contamination of water lines, in particular, the water lines of a dental unit. The antimicrobial formulation of the present invention includes, but is not limited to colloidal silver, from between about 0.001 to 2 percent by weight and colloidal copper, from between about 0.001 to about 10 percent by weight. The copper protects teeth against decay, dental caries and plaque formation. Hence, the formulation may be useful to treat tooth decay alone or in combination with other compounds such as stannous fluoride and sodium fluoride.

Copper containing dental cements have been shown to demonstrate germicidal activity. See e.g., M. Dumas and M. Blush. Bacteriocidal Effects of Copper Cements: A Review of the Literature, Tufts Dental Outlook, 27(2): 1-5 (1954). Similarly, silver has also been shown to have antimicrobial properties. See e.g., Hill et al., Relative Efficiency of Germicidal Cements, The Journal of the American Dental Association, 21(3): 1565-1571 (1934).

The antimicrobial formulation may be provided in a powder or solid form. The formulation may also be mixed with other compositions and substances described herein and/or used in combination with other compositions and substances.

For example, iron or iron compounds may be added to further enhance the antimicrobial effect of the subject formulation. In dental indications, iron increases immune response and is a primary substance in helping to prevent chronic infection and pain in teeth and gums. Iron may be present in concentrations ranging from about 0.05 to 3 percent by weight in the form of ferric oxide, for example.

The antimicrobial formulations and blends may also be mixed with other compositions or used in combination with other compounds or dental compositions. For example, the formulation may be mixed with calcium hydroxide and placed on a tooth to protect against or retard decay. The formulation may also be combined with a composite resin and placed as a base. The formulation may also be used in combination with fluoride compounds including but not limited to sodium fluoride, and stannous fluoride. Bismuth has also been shown to be effective in dental compositions containing copper ions. See e.g, M. Dumas and M. Blush. Bacteriocidal Effects of Copper Cements: A Review of the Literature, Tufts Dental Outlook, 27(2): 1-5 (1954).

The present invention provides formulations and blends that are highly antimicrobial, non-toxic, biocompatible, non-irritating to dental tissues, are color-fast, easy to prepare, have adequate setting time, have superior mechanical properties, promote secondary dentin formation and add years of longevity to fillings, castings and endodontic posts.

The formulations of the present invention are useful in combination with a wide variety of dental compositions and treatments including, but not limited to, resins, fillings, bases, periodontal packs, cements such as red copper cement, sealers such as root canal sealers or root crack sealers, calcium hydroxide preparations for direct pulp caps, compomer/glass ionomer products, light curing adhesives, two-part bonding adhesives, core build up materials, pit and fissure sealant, composite restorative materials, periodontal dressings, toothpastes, and chewable tablets.

Antibacterial formulation compositions may vary depending on application. For example, in compomer/glass ionomer products colloidal copper may be present in about 0.5% by weight and colloidal silver in about 0.25% by weight. For light curing or two-part bonding adhesives colloidal copper may be present in about 0.25% by weight and colloidal silver in about 0.25% by weight. In core build up materials colloidal copper may be present in about 0.75% by weight and colloidal silver in about 0.25% by weight. Pit and fissure sealants may include about 2% colloidal copper by weight and about 1% colloidal silver by weight. Composite restorative materials may contain about 0.5% colloidal copper by weight and about 0.25% colloidal silver by weight. In periodontal dressings there may be about 8% colloidal copper by weight and about 1% colloidal silver by weight. Such dressings may also include zinc oxide (ZnO) and the formulation made into a cement with eugenol liquid.

Antimicrobial formulations of the present invention may be incorporated into chewable tablet form. Such a tablet may be useful in the field, for example, by soldiers where normal hygiene practice may be particularly cumbersome or impractical.

Additionally, a kit may be provided with an antimicrobial formulation having colloidal silver, from between about 0.001 to 2 percent by weight and colloidal copper, from between about 0.001 to about 10 percent by weight are packaged separately along with a dental varnish, stannous fluoride or sodium fluoride.

Other applications include, but are not limited to time release antimicrobial chips, which include a mixture of colloidal silver and colloidal copper in a ratio of about 3:1. The mixture may be electroplated on a base material, such as carbon bone or other carrier material. Such chips may use a base material that is biocompatible for dental applications, for example. Other base materials may be incorporated in composite materials for wear-dependent release of the antimicrobial formulation.

### EXAMPLES

The following examples are included to demonstrate particular embodiments of the present invention. It should be appreciated by those of skill in the art that the methods disclosed in the example that follows merely represent exemplary embodiments of the present invention. However, those of skill in the art should, in light of the present disclosure, appreciate that many changes can be made in the specific embodiments described and still obtain a like or similar result without departing from the spirit and scope of the present invention.

### Example 1

### Antimicrobial Formulation Useful For Preparing Red Copper Cement.

| | Percentage by weight |
|---|---|
| silica | 4 |
| magnesium oxide | 6 |
| zinc oxide | 73 |
| bismuth subnitrate | 5 |
| ferric oxide | 3 |
| Colloidal copper | 2 |
| silver chloride | 1 |

Silica, magnesium, zinc oxide, bismuth sub nitrate and ferric oxide are first calcined by mixing the dry powders in a sealed mixer. The mixture is then placed in an airtight porcelain crucible and heated to 1,050° C. for three hours. The result is a fine powder said to be more medicinal and less toxic than an uncalcined equivalent.

The silver chloride is then made finer by grinding or crushing in a low oxygen, low light environment to protect the compound from oxidation and color change. Following this procedure the silver chloride powder is mixed uniformly along with the above calcined base in the sealed mixer. The colloidal copper may be added at the same point and thoroughly mixed, leaving a red powder which completes the formulation.

This formulation may be antimicrobial, highly biocompatible, color fast, easy to mix, have adequate setting time, may be easy to clean after setting, add years of longevity to fillings, castings and endodontic posts and promote secondary dentin formation. Red copper cement can directly impact and reduce tendencies to the pathological patterns and tissues on a bio-energetic functions level including follicular cysts, dental fistula, dental foot granuloma, fundus abscess, gingival sulcus, ulcerous gigivitis, maxillary ostitis, exudative ostitis, sclerotic ostitis, pepto-streptococci, borrelia burgdorfer, gangrenous pulpa, acute pulpitis, and caries.

This formulation may be used as a powder or mixed with an aqueous or non-aqueous medium. This formulation may be heated at high temperatures for a prolonged period of time in order to calcinate the formulation used to make red copper cement. The formulation of the present invention is particularly useful in preventing and treating dental decay alone or in combination with fluoride compounds such as stannous fluoride and sodium fluoride between about 0.05 weight percent and 5 weight percent.

Also, the red copper powder may be mixed with a combination of 400 ppm ethyl ether anhydrous and/or COPALITE WE® (a dental varnish without chloroform) to treat fungal toenails.

### Example 2

### Root Canal Sealer.

| | Percentage by weight |
|---|---|
| zinc oxide | 4 |
| partially hydrogenated rosin | 25 |
| bismuth subcarbonate | 14 |
| barium sulfate | 11 |
| sodium borate | 2 |
| cuprous oxide | 7 |
| silver chloride | 1 |

This formulation may be mixed in with combination of 400 ppm ethyl ether anhydrous and/or Copalite WE® (a dental varnish without chloroform) and may be used to seal root canals when eugenol liquid is the medium.

### Example 3

### White Cement Formulation

| | |
|---|---|
| silica | 4% |
| magnesium oxide | 6% |
| zinc oxide | 81% |
| bismuth subnitrate | 5% |
| colloidal copper | 0.001% |
| colloidal silver | 0.001% |
| titanium dioxide | 3% |

An antimicrobial formulation may be included in a white cement formulation as shown above. The colloidal copper and colloidal silver are added to the formulation after calcinations of the remaining ingredients, as described above.

Example 4 shows a toothpaste formulation incorporating the antimicrobial formulation of the present invention. One skilled in the art will recognize that the reactive alkali metals (calcium, magnesium, sodium, and potassium) are present as salts and that the weight percent of these metals are obtained by elemental analysis. Other elements may be present either in their elemental form or as salts. For example, zinc may be present in oxide form, ZnO.

### Example 4

### Toothpaste Formulation

| Ingredient | Min % by wt. | Max % by wt. |
|---|---|---|
| xylitol | 30 | 36 |
| calcium | 36 | 39 |
| magnesium | 20 | 24.3 |
| zinc | 0.025 | 0.0755 |
| copper | 0.010 | 0.012 |
| manganese | 0.05 | 0.09 |
| sodium | 0.02 | 0.0755 |
| potassium | 0.01 | 0.0370 |
| silver | 0.02 | 0.0378 |
| bismuth | 0.02 | 0.0755 |
| silica | 0.11 | 0.2987 |

Example 5

The formulation may be used to treat dental water lines.

The formulation may be used as a 100% silver colloidal powder mixed into an aqueous medium between 23 to 36 ppm. The formulation is introduced into the water lines of a dental unit and the liquid is allowed to remain undisturbed overnight. The next morning, the lines are rinsed for 20 seconds, Commercial test kits may be used to indicate that the water lines of the dental unit were purged of microbes.

### Example 6

### Efficacy of Dental Cement in Biofilm Prevention in a Stagnant System.

The following results demonstrate the efficacy of dental cement coupons containing antimicrobial formulations with colloidal copper and colloidal silver. The CBE with 4 types of standard-sized coupons (labeled GLS, RCC, WC, and WC+). The tests described below are performed in stagnant test tubes.

### Growth Curve Study

A growth curve study of Streptococcus mutans ATCC 25175 and Lactobacillus paracasei was completed in order to determine optimal media concentration and time necessary for the organisms to reach log phase. S. mutans was grown anaerobically in full-strength Brain Heart Infusion (BHI) broth and L. paracasei was grown aerobically in full strength Lactobacilli MRS broth. Both cultures were incubated at 37° C. Based on the growth curve study, it was determined that S. mutans reaches log phase after 22 hours of anaerobic incubation in BHI broth at 37° C. L. paracasei reach log phase after 46 hours of aerobic incubation Lactobacilli MRS broth at 37° C.

### Stagnant Biofilm Study

Eight coupons of each type were placed in glass scintillation vials and autoclaved. Sterile BHI broth at a concentration of 37 mg/L and sterile Lactobacilli MRS broth at a concentration of 55 mg/L was added to appropriate vials. The tubes were incubated at room temperature for 48 hours. The tubes were inoculated with 100 μl of S. mutans or L. paracasei grown for the appropriate amount of time at a concentration 10³ —10⁴ CFU/ml. Vials inoculated with S. mutans were incubated anaerobically for 48 hours at room temperature. Vials inoculated with L. paracasei were incubated for 48 hours aerobically at room temperature.

At the end of the 48 hour incubation, the coupons were removed from the vials, scraped to remove biofilm. The biofilm samples were disaggregated via homogenization. The samples were diluted and plated on tryptic soy agar. S. mutans samples were incubated anaerobically for 60 hours at 37° C. L. paracasei samples were incubated aerobically for 60 hours at 37° C.

As is apparent in Table 1, all coupon types completely retarded biofilm colonization with the exception of GLS coupons challenged with Lactobacillus in MSR media, and PC coupons challenged with Lactobacillus in MSR media. No colonization was observed on any other coupon samples.

TABLE 1
Test tube designation, inoculum, media and average colonization.

| Tube | Coupon | | | Average Colonization |
|---|---|---|---|---|
| Numbers | Type^{a} | Innoculum | Media | (CFU/cm²) |
| 1-3 | GLS | S. mutans | BHI | 0 |
| 4-6 | GLS | Lactobacillus | MRS | 684 |
| 7 | GLS | none | BHI | 0 |
| 8 | GLS | none | MRS | 0 |
| 9-11 | RCC | S. mutans | BHI | 0 |
| 12-14 | RCC | Lactobacillus | MRS | 0 |
| 15 | RCC | none | BHI | 0 |
| 16 | RCC | none | MRS | 0 |
| 17-19 | WC | S. mutans | BHI | 0 |
| 20-22 | WC | Lactobacillus | MRS | 0 |
| 23 | WC | none | BHI | 0 |
| 24 | WC | none | MRS | 0 |
| 25-27 | WC+ | S. mutans | BHI | 0 |
| 28-30 | WC+ | Lactobacillus | MRS | 0 |
| 31 | WC+ | none | BHI | 0 |
| 32 | WC+ | none | MRS | 0 |
| 33-35 | PC | S. mutans | BHI | 0 |
| 36-38 | PC | Lactobacillus | MRS | 210 |
| 39 | PC | none | BHI | 0 |
| 40 | PC | none | MRS | 0 |

| ^{a}Cement | | | |
|---|---|---|---|
| Coupons: | Product | Lot# | Manufacturer |
| GLS | Fuji I Glass Ionomer | 0402051 | GC America |
| | Cement with Fluoride | | |
| RCC | DOC'S BEST Red Copper | A2920-9 | Cooley & |
| | Cement | | Cooley Ltd |
| WC | DOC'S BEST White | i3810-6 | Cooley & |
| | Copper Cement | | Cooley Ltd |
| WC+ | White Copper + | CS-11-04 | CopperDENTX |
| | formula in Cement | | |
| PC | Durelon Cement | 166317 | 3M ESPE |
| | (Polycarbonate Cement) | | |

Advantageously, in dental indications, copper increases local and general immunity and prohibits chronic inflammation and infection in tissues associated with local dental procedures. Copper supports tissue metabolism and enhancing detoxification of the oral cavity including the teeth and bone tissues and serves as a germicidal agent. Incorporation of copper and silver colloidal materials in dental building materials provides lasting germicidal protection in an environment under constant moist conditions where fluoride salts have short half life. Uses of the formulation described herein may be useful in the treatment of tooth decay when used in combination with standard reconstructive dental surgery.

Although the invention has been described with reference to specific embodiments, these descriptions are not meant to be construed in a limiting sense. Various modifications of the disclosed embodiments, as well as alternative embodiments of the invention will become apparent to persons skilled in the art upon reference to the description of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

It is therefore, contemplated that the claims will cover any such modifications or embodiments that fall within the true scope of the invention.

What is claimed is:

1. An antimicrobial formulation for dental applications comprising:
(a) colloidal silver, from between about 0.001 to 2 percent by weight; and
(b) colloidal copper, from between about 0.001 to about 10 percent by weight

2. The antimicrobial formulation of claim 1, further comprising an iron compound from about 0.05 to 3 percent by weight, wherein the iron compound is ferric oxide.

3. The antimicrobial formulation of claim 1, further comprising one or more compounds selected from the group consisting of silica, magnesium oxide, titanium oxide, zinc oxide, partially hydrogenated rosin, barium sulfate, bismuth subnitrate, xylitol, eugenol, and sodium borate.

4. The antimicrobial formulation of claim 1, wherein said formulation is incorporated in at least one selected from the group consisting of a red copper cement, a white cement, compomer/glass ionomer products, light curing adhesives, two-part bonding adhesives, core build up materials, pit and fissure sealant, composite restorative materials, periodontal dressing, a toothpaste, and a chewable tablet.

5. A method of treating microorganisms on a tooth comprising the step of administering to a patient in need thereof an effective amount of the antimicrobial formulation of claim 1 alone or in combination with a dental varnish, stannous fluoride, or sodium fluoride.

6. A red cement formulation comprising:
(a) about 4% by weight silica;
(b) about 6% by weight magnesium oxide;
(c) about 73% by weight zinc oxide;
(d) about 5% by weight bismuth subnitrate;
(e) about 3% by weight ferric oxide;
(f) about 2% colloidal copper; and
(g) about 1% by weight silver chloride.

7. A kit comprising:
(1) an antimicrobial formulation comprising:
(a) colloidal silver, from between about 0.01 to 2 percent by weight; and
(b) colloidal copper, from between about 0.05 to about 10 percent by weight; and
(2) a dental varnish, stannous fluoride or sodium fluoride.

8. A white cement formulation comprising:
about 4% silica;
about 6% Magnesium oxide;
about 81% zinc oxide;
about 5% bismuth subnitrate;
about 0.001% colloidal copper;
about 0.001% colloidal silver; and
about 3% titanium dioxide.

9. A time release antimicrobial chip comprising a mixture of colloidal silver and colloidal copper in a ratio of about 3:1, wherein said colloidal silver and said colloidal copper are electroplated on a base material.

10. A method of preventing tooth decay comprising the step of administering to a patient in need thereof an effective amount of the antimicrobial formulation of claim 1 alone or in combination with a dental varnish, stannous fluoride, or sodium fluoride.

11. An antimicrobial formulation for dental applications comprising at least one selected from the group consisting of:
(a) colloidal silver, from between about 0.001 to 2 percent by weight ;and
(b) colloidal copper, from between about 0.001 to about 10 percent by weight.

12. A toothpaste comprising an antimicrobial formulation of claim 1 or claim 11.
